(19) United States
(12) Patent Application Publication
(10) Pub. No.: US 2005/0112395 A1
(43) Pub. Date: May 26, 2005

(54) Porous body and method for producing the same

(76) Inventor: Akira Harada, Ina-si (JP)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/968,450

(22) Filed: Oct. 19, 2004

(30) Foreign Application Priority Data
Nov. 21, 2003 (JP) 2003-391885

(51) Int. Cl. A61F 2/28

(52) U.S. Cl. 428/548

(57) ABSTRACT

A cylindrical porous body composed of a number of base particles adhering to one another with an adhesion material having a lower melting point than the melting point of the base particle, in which an wire mesh is attached to an internal surface thereof.

### BACKGROUND OF THE INVENTION

1. Field of the Invention

The present invention relates to a porous body useful for an inflator for an airbag of a vehicle, and a method for producing the same.

2. Description of the Related Art

Conventionally, a variety of porous bodies and methods for producing the same have been known. For instance, there is known a cylindrical porous body, which is produced by compressing a material knitted out of fine metal wires by a weaving machine for fine metal wire rods so that it forms a cylindrical shape, and a method for producing the same (refer to Japanese Patent Application Laid-Open No. Hei 7-285412, as an example). Also, there is known a cylindrical porous body, which is produced by cutting a metal plate into rhombic shapes to form a coil shape, and cutting and winding up the same to form the cylindrical porous body, and a method for producing the same (expanding processing) (refer to Japanese Patent Application Laid-Open No. 2002-249017, as an example). Further, in the other case, there is known a porous body produced by generating thousands of holes by foaming a metal and a method for producing the same. Furthermore, there is known a porous body produced by molding and sintering spherical-shaped powders, and a method for producing the same (refer to Japanese Patent Application Laid-Open No. 2002-331235, as an example).

However, the above-listed conventional techniques have problems as will be described below. In the production of the porous body disclosed in the Japanese Patent Application Laid-Open No. Hei 7-285412, there is a limit on the girth of the metal wires weavable by the machine for metal wire rods. Therefore, when used under high pressure and aeration, the porous body is unable to resist the high pressure. Besides, it requires too much time to weave the metal wires, resulting in high production cost.

In the production of the porous body disclosed in the Japanese Patent Application Laid-Open No. 2002-249017, there is a problem of requiring many processes in the expanding processing, resulting in high production cost. Also, in the production of the porous body produced using a foam metal, there are problems of low pressure resistance and difficulty in low cost production, while exhibiting its capability of producing the porous body of a high quality having fine holes controlled in diameter and length.

Further, the porous body disclosed in the Japanese Patent Application Laid-Open No. 2002-331235 has a problem of low pressure resistance. Furthermore, when producing the porous body by sintering, a process for shaping the outer shape of the porous body into a predetermined shape is required. That is, a molding step using a metal mold is required in general, resulting in high production cost. The sintering without using the metal mold is conceivable, yet, in that case, there arise problems of slow sintering and low pressure resistance of the porous body. On top of this, a technique, which performs sintering after shaping without using the metal mold and following cutting out of the porous body into a desired shape, is also conceivable. Notwithstanding the above, in view of the production cost, the cutout process requires higher cost than that of the molding process using the metal mold. It is therefore impossible to produce the porous body at low cost, leaving a problem. In addition, further improvement in cooling performance, cleansing performance, or sound-deadening performance has been expected.

In consideration of the above-described problems, with an aim to provide a porous body being highly pressure resistant and manufacturable at low cost and a method for producing the same, the present inventor has developed a porous body which is composed of a number of base particles caused to adhere to one another by an adhesion material having a melting point lower than the melting point of the base particle.

Notwithstanding the above, the porous body is still expected improvement further in view of production efficiency and performance. Specifically, after the heating of the base particles and the adhesion material in a cylindrical molding container in the production process, it is desired that the resulting molded product can be removed more easily. In view of the performance, the porous body is expected to have a long operating life as a filter that passes a fluid from the center of the cylindrical porous body to the lateral surface thereof as smooth as possible. Incidentally, as one approach to remove the molded product, which is obtained after the heating, from the cylindrical molding container with ease, an insertion of a ceramic sheet into the cylindrical molding container is also conceivable, whereas, the production cost thereby increases.

### SUMMARY OF THE INVENTION

Accordingly, an object of the present invention is to facilitate removal of a porous body and to improve an operating life of the porous body in view of filtering performance.

In order to accomplish the above-stated object, the present invention is a cylindrical porous body composed of a number of base particles caused to adhere to one another by an adhesion material having a melting point lower than the melting point of the base particle, in which a wire mesh is attached to an internal surface of the porous body. Therefore, after the mixture of the base particles and the adhesion material is heated, the mixture is fixed only to the wire mesh without fixing to the central axis of a cylindrical molding container. Additionally, when a heated air passes through the cylindrical porous body from the center thereof, the heated air is cooled down while passing through the wire mesh, so that the adhesion material of the porous body is difficult to melt. Hence, the porous body can be prevented from a clogging.

Another present invention is the porous body further having the wire mesh attached to a lateral surface thereof. Therefore, the porous body can be prevented from damaging, when a high-temperature and high-pressure blast arises inside the porous body.

Further, still another present invention is the porous body in which the diameter of a wire of the wire mesh is within the range of 0.3 mm or more and 1.0 mm or less, preferably, within the range of 0.5 mm or more and 0.8 mm or less. By designing the wire mesh to have a thickness of 0.3 mm or more, preferably, 0.5 mm or more, resistance properties to a high-temperature and high-pressure blast is enhanced. Meanwhile, by designing the wire mesh to have a thickness of 1.0 mm or less, preferably, 0.8 mm or less, the processing of the wire mesh and the attachment of the wire mesh to the central axis of the cylindrical molding container are facilitated, and the production cost of the wire mesh can be reduced as well.

Furthermore, still another present invention is the porous body of which wire mesh is an expanded metal. The porous body is thereby enabled to have lesser projections and recessions on the internal surface thereof. On the back of this, the porous body can ensure effective volume of the internal space thereof. Additionally, pressure resistance to the high-temperature and high-pressure blast is further enhanced.

Furthermore, still another present invention is the porous body in which a shorter diagonal of each of rhombic holes of the expanded metal has a length passing no base particle therethrough and within the range of 0.3 mm or more and 2.0 mm or less. Of the diagonals of each rhombic hole of the expanded metal, the shorter diagonal has a length of 0.4 mm or more, which does not hinder air permeability of the porous body. Also, the diagonal is designed to have a length of 2.0 mm or less, so that base particles commonly used for a part of an inflator for an airbag do not pass through the rhombic hole. Further, the expanded metal having 2.0 mm or less in length of the shorter diagonal requires no customization, serving to lower the production cost of the porous body.

Furthermore, still another present invention is the porous body in which the adhesion material is metal. Therefore, the adhesion material enters into boundary surfaces between the base particles securely, bringing strong structure, so that pressure resistance of the porous body can be enhanced.

Furthermore, still another present invention is the porous body in which the base particle is iron and the adhesion material is copper. Therefore, while heating the iron particles being the base particles, the copper disperses in a liquid manner over the boundary surfaces and surfaces of the iron particles, and thereby the surface areas become larger than those of the porous body composed only of the iron particles to the extent of protrusion and recession formed by copper or the protrusion and recession formed by iron and copper. Accordingly, the porous body can be improved in pressure resistance, cooling performance, cleaning performance, and sound-deadening performance. In addition, copper is a relatively low-cost metal material, enabling cost reduction for producing the porous body.

Furthermore, still another present invention is a method for producing a porous body, which includes the steps of: mixing a number of base particles composing the porous body and an adhesion material causing the base particles to adhere to one another, the adhesion material having a lower melting point than the melting point of the base particle; attaching a wire mesh to a lateral surface of a central axis of a cylindrical molding container for molding to form a cylindrical shape; heating a mixture, which is obtained by the mixing step, in a state being in the cylindrical molding container after the attachment step so that the base particles adhere to one another with the adhesion material, and removing a molded product from the cylindrical molding container. Therefore, after the mixture of the base particles and the adhesion material is heated, the mixture is fixed only to the wire mesh without fixing to the central axis of the cylindrical molding container. Additionally, when a heated air passes through the cylindrical porous body from the center thereof, the heated air is cooled down while passing through the wire mesh, so that the adhesion material of the porous body is difficult to melt. Hence, the porous body can be prevented from a clogging.

Furthermore, still another present invention is the method for producing the porous body in which the attachment step of the wire mesh is the step of attaching the wire mesh also to an internal surface of the central axis of the cylindrical molding container. Therefore, the porous body can be prevented from damaging, when a high-temperature and high-pressure blast arises inside the porous body.

Furthermore, still another present invention is the method for producing the porous body in which the diameter of a wire of the wire mesh is within the range of 0.3 mm or more and 1.0 mm or less, preferably 0.5 mm or more and 0.8 mm or less. By designing the wire mesh to have a thickness of 0.3 mm or more, preferably, 0.5 mm or more, resistance properties to a high-temperature and high-pressure blast is enhanced. Meanwhile, by designing the wire mesh to have a thickness of 1.0 mm or less, preferably, 0.8 mm or less, the processing of the wire mesh and the attachment of the wire mesh to the central axis of the cylindrical molding container are facilitated, and the production cost of the wire mesh can be reduced as well.

Furthermore, still another present invention is the method for producing the porous body in which the wire mesh is an expanded metal. The produced porous body is thereby enabled to have lesser projections and recessions on the internal surface thereof. On the back of this, the porous body can ensure effective volume of the internal space thereof. Additionally, pressure resistance to the high-temperature and high-pressure blast is further enhanced.

Furthermore, still another present invention is the method for producing the porous body in which the expanded metal has a number of rhombic holes and a shorter diagonal of the diagonals of the hole has a length passing none of the base particle therethrough and within the range of 0.3 mm or more and 2.0 mm or less. Of the diagonals of each rhombic hole of the expanded metal, the shorter diagonal has a length of 0.4 mm or more, which does not hinder air permeability of the porous body. Also, the diagonal is designed to have a length of 2.0 mm or less, so that base particles commonly used for a part of an inflator for an airbag do not pass through the rhombic hole. Further, the expanded metal having 2.0 mm or less in length of the shorter diagonal requires no customization, serving to lower the production cost of the porous body.

Furthermore, still another present invention is the method for producing the porous body in which the adhesion material is metal. Therefore, the adhesion material enters into boundary surfaces between the base particles securely, bringing strong structure, so that pressure resistance of the porous body can be enhanced.

Furthermore, still another present invention is the method for producing the porous body in which the base particle is iron and the adhesion material is copper. Therefore, while heating the iron particles being the base particles, the copper disperses in a liquid manner over the boundary surfaces and surfaces of the iron particles, and thereby the surface areas become larger than those of the porous body composed only of the iron particles to the extent of protruding and recessed portions formed by copper or the protruding and recessed portions formed by iron and copper. Accordingly, the porous body can be improved in pressure resistance, cooling performance, cleaning performance, and sound-deadening performance. In addition, copper is a relatively low-cost metal material, enabling cost reduction for producing the porous body.

According to the present invention, the porous body can be removed with ease and a longer operating life in filtering performance can be realized.

### BRIEF DESCRIPTION OF THE DRAWINGS

FIG. 1 is a view showing steps before a heating step in a method for producing a porous body according to a first embodiment of the present invention;

FIG. 2 is a view schematically showing a heating unit used in respective embodiments of the present invention for heating the mixture of base particles and an adhesion material;

FIG. 3 is a view showing a removing step for taking out the porous body that is a molded product from a container after a heating step in the method for producing the porous body according to the first embodiment of the present invention;

FIG. 4 is a flowchart showing a production process flow of the method for producing the porous body according to the first embodiment of the present invention;

FIG. 5 is a view showing the porous body after the removing step shown in FIG. 4, viewing from the direction of a hole;

FIG. 6 is a view showing a part of an expanded metal shown in FIG. 3;

FIGS. 7A and 7B are views schematically showing a change in copper powders that exist between iron particles in the heating step shown in FIG. 4, in which, respectively, FIG. 7A shows a state before heating and FIG. 7B shows a state during the heating, in which coppers existing on the surfaces of the upper and lower iron particles are about to enter into contact portions of the iron particles substantially in the center of a space formed by four iron particles;

FIG. 8 is a view schematically showing, in an enlarged illustration, an existing state of the iron particles and the copper of the porous body obtained after the cooling step shown in FIG. 4;

FIGS. 9A and 9B are views comparatively showing the microstructures of the porous body obtained by sintering the iron particles and the porous body obtained through the production process shown in FIG. 4, in which, FIG. 9A shows the microstructure of the porous body obtained by sintering the iron particles, and FIG. 9B shows the microstructure of the porous body obtained through the production process shown in FIG. 4, respectively;

FIG. 10 is a view showing how a blast arising from an explosion in the hole of the porous body obtained through the production process in FIG. 4 passes through the space inside the porous body together with a partial enlarged illustration thereof;

FIG. 11 is a view showing steps before a heating step in the method for producing the porous body according to a second embodiment of the present invention;

FIG. 12 is a view showing a removing step for taking out the porous body that is a molded product from the container after the heating step in the method for producing the porous body according to the second embodiment of the present invention;

FIG. 13 is a view showing how the blast arising from the explosion in the hole of the porous body obtained in the method for producing the porous body according to the second embodiment of the present invention passes through the space inside the porous body;

FIG. 14 is a view for explaining a part of the production process for a solid columnar porous body according to a third embodiment of the present invention;

FIG. 15 is a flowchart showing the production process for the solid columnar porous body according to the third embodiment of the present invention;

FIG. 16 is a view showing the solid columnar porous body obtained through the production process shown in FIG. 15;

FIG. 17 is a view showing, in a partially enlarged illustration, how the blast passes through from one planar surface of the solid columnar porous body shown in FIG. 16; and

FIG. 18 is a flowchart showing the production process completed by attaching the expanded metal on the solid columnar porous body after cooling.

### DETAILED DESCRIPTION OF THE PREFERRED EMBODIMENTS

Hereinafter, respective embodiments of a porous body and a method for producing the same according to the present invention will be described with reference to the drawings.

First, description will be given in respect to materials for the porous body used in common in the following respective embodiments.

The materials for the porous body are roughly classified into a base particle to compose a parent body of the porous body and an adhesion material for causing the base particles to adhere to one another. Incidentally, in the respective embodiments below, for the purpose of describing an adhesion of metals to one another as examples, “welding”, which is a subordinate concept of the adhesion, will be used. In the course of a heating step, which is one of the steps of the production process for the porous body, the adhesion material melts and goes into a liquid state to cause the base particles to adhere to one another on boundary faces of the base particles. This explains why the adhesion material is required to have a lower melting point than that of the base particle, as one requirement, in producing the porous body.

In the following respective embodiments, for the base particles, iron particles are used, and for the adhesion material, a copper brazing material or a nickel brazing material is used. The iron melts approximately at 1535° C., the nickel melts approximately at 1450° C., and the copper melts approximately at 1083° C. In addition, since the brazing materials are used as adhesion materials, the iron particles can be brazed approximately at 1100° C. to 1150° C. when the copper brazing material (99.9 wt % Cu contained) is used. Meanwhile, when the nickel brazing material (a nickel alloy containing 4 to 5 wt % Si, approximately 3 wt % B, and approximately 10 wt % Cr) is used, the iron particles can be brazed at 925° C. to 1175° C., which is lower than the melting point of nickel. Hence, it is possible to produce the porous body made of iron by causing a number of iron particles to adhere to one another by the adhesion material having a lower melting point than that of the iron.

The iron particles used in the respective embodiments are spherical type iron particles having 1.0 mm in diameter on average. The copper powders used in the respective embodiments have 0.05 mm in diameter on average. However, the sizes of the iron particle and the copper powder are not limited thereto, and the sizes are variable in accordance with usages or production methods of the porous body. Note that the copper can be provided in various forms such as a powder, a liquid body, and the like.

### First Embodiment

Firstly, a first embodiment of the present invention will be described. In the first embodiment, description will be provided for a porous body, which is obtained by winding a cylindrical expanded metal as one aspect of a wire mesh around the side surface of the central axis of a cylindrical molding container, and by inputting and subsequently heating a mixture of iron particles as base particles and copper brazing material containing copper powders as an adhesion material, as well as a method for producing the same. Note that, in the drawings below, there are shown only copper powders contained in the copper brazing, restraining to illustrate any liquid material except the copper powders.

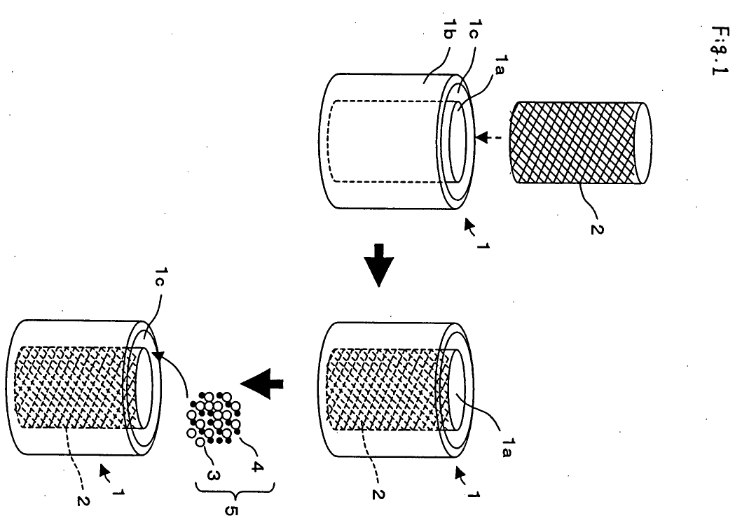

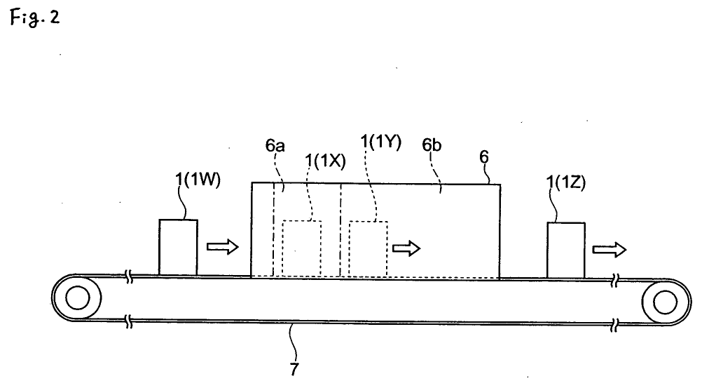

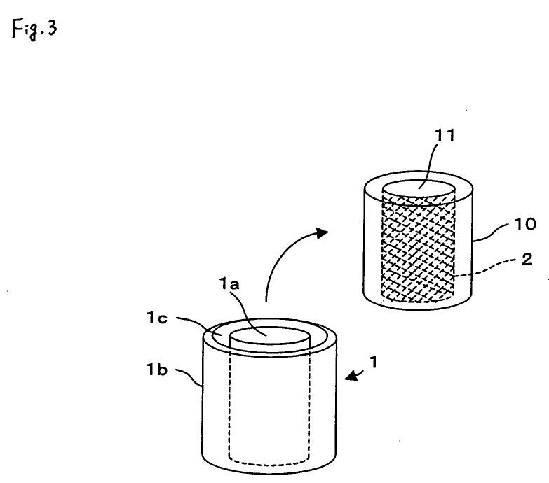

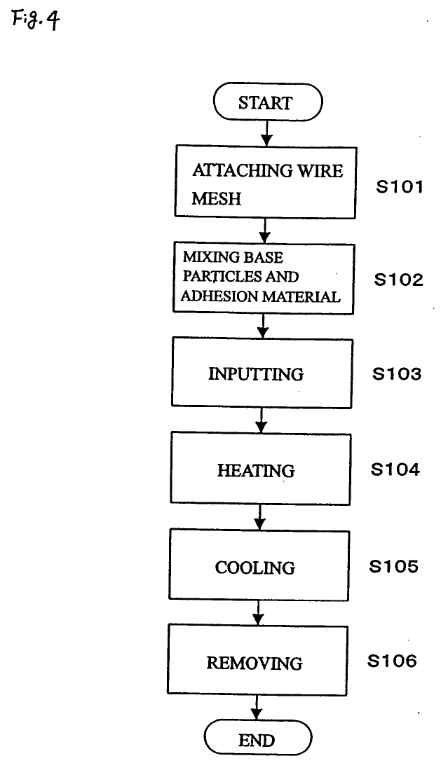

FIG. 1 is a view showing, in the production process of the porous body, those steps before a heating step. FIG. 2 is a view schematically showing a heating unit for heating the mixture of the base particles and the adhesion material. FIG. 3 is a view showing a removing step to take out the porous body that is a molded product from the container. Further, FIG. 4 is a flowchart showing a production process flow for producing the porous body according to the first embodiment.

In order to produce the porous body of a cylindrical shape, a cylindrical molding container as shown in FIG. 1 (hereinafter referred to as a “bottomed cylindrical sleeve container”) 1 is used. The bottomed cylindrical sleeve container 1 is constituted by a central axis that stands at the center of the container (hereinafter referred to as a “core section”) 1a, and an outer side section 1b. Between the core section 1a and the outer side section 1b of the container, there is a cylindrical space 1c. When performing a molding after inputting the iron particles as the base particles and the copper brazing material as the adhesion material into the cylindrical space 1c, a molded product having the shape of the cylindrical space 1c is obtained.

As shown in the flowchart in FIG. 4, the production process of the cylindrical porous body includes: an attachment step (step S101) for attaching the expanded metal as one aspect of the wire mesh, a mixing step (step S102) for mixing the iron particles as the base particles and the copper brazing material as the adhesion material, an inputting step (step S103) for inputting the mixed materials into the bottomed cylindrical sleeve container 1, a heating step (step S104) for inputting the bottomed cylindrical sleeve container 1 having the mixed materials into a heating furnace and heating them, a cooling step (step S105) for cooling the molded product obtained from the heating step, and a removing step (step S1106) for taking out the molded product from the bottomed cylindrical sleeve container 1.

In the attachment step S101, the core section 1a of the bottomed cylindrical sleeve container 1 is surrounded by an expanded metal 2 as shown in FIG. 1. In the mixing step S102, iron particles 3 and a copper brazing material 4 are mixed by an unshown mixing machine. In the inputting step S103, as shown in FIG. 1, a mixture 5 of the iron particles 3 and the copper brazing material 4 is inputted into the cylindrical space 1c in the bottomed cylindrical sleeve container 1 of which core section 1a is in a state surrounded by the expanded metal 2.

It should be noted that the iron particles 3 and the copper brazing material 4 can be inputted into the bottomed cylindrical sleeve container 1 first, and mixed thereafter. The production process in that case comes to a flow that replaces step S102 and step S103 in FIG. 4 with each other.

After the inputting step, the bottomed cylindrical sleeve container 1 having the mixture 5 therein is inputted into a heating furnace 6 as shown in FIG. 2. The heating step S104 and the cooling step S105 are carried out by moving the bottomed cylindrical sleeve container 1 having the mixture 5 therein in the direction of outline arrows toward a position 1W, a position 1×, a position 1Y, and a position 1Z on a belt conveyer 7 capable of moving the bottomed cylindrical sleeve container 1 (in FIG. 2, the bottomed cylindrical sleeve container 1 at each position is denoted by 1 (1W), 1 (1X), 1 (1Y), 1 (1Z)).

In the heating furnace 6 according to the first embodiment, there exists a heating section 6a in the vicinity of an inlet and there is disposed a cooling section 6b at an outlet side. The highest temperature of the heating section 6a inside the heating furnace 6 is approximately 1100° C. This temperature is an appropriate temperature for the copper powders of the copper brazing material 4 to melt down to be a molten state, and thereby the iron particles 3 are caused to adhere to one another. Note that the temperature of the heating furnace 6 can be changed adequately depending on kinds of the adhesion materials. Besides, inside the heating furnace 6 is placed under a condition of a reducing atmosphere gas (for example, hydrogen), so that oxide films on surfaces of the iron particles 3 can be reduced.

According to the first embodiment, the belt conveyer 7 is continuously in operation, and therefore the bottomed cylindrical sleeve container 1 is heated and then cooled down while moving in the direction of the arrows. Alternatively, it is also possible to design such that the contents of the bottomed cylindrical sleeve container 1 is cooled down by being left as it is at the position 1Z in FIG. 2 by halting the belt conveyer 7 for a predetermined time. Further, the heating furnace 6 having no cooling section 6b is also acceptable. In that case, by moving the bottomed cylindrical sleeve container 1 to outside the belt conveyer 7 after the heating, the continuous production of the porous bodies is possible while operating the belt conveyer 7 without the halt.

After the cooling step S105, as shown in FIG. 3, a porous body 10, which is a molded product, is removed from the bottomed cylindrical sleeve container 1. On an inside wall of a hole 11 of the removed porous body 10, the expanded metal 2, which is previously set so as to surround the core section 1a of the bottomed cylindrical sleeve container 1, is fixed. This is because, in the course of the heating step and the cooling step, the mixture 5 is fixed to the expanded metal 2 while heated to shrink. The expanded metal 2 is previously set to surround the core section 1a in a loose manner, so that the porous body 10 can be removed from the bottomed cylindrical sleeve container 1 together with the expanded metal 2 with ease. Accordingly, a removing work of the porous body 10 is facilitated, and further, a possible damage that the porous body 10 is to suffer in the production process can be reduced since no unnecessary force is required when removing the porous body 10.

FIG. 5 is a view showing the porous body 10 after the removing step by viewing from the direction of the hole 11.

The expanded metal 2 fixing to the inside wall of the hole 11 has an optional board thickness X within the range of 0.5 mm or more and 0.8 mm or less. The expanded metal 2 is thus designed to have the board thickness X of 0.5 mm or more, so that resistance properties against a high temperature and high pressure blast can be enhanced for example when the porous body 10 is used as a part of an inflator for an airbag. Meanwhile, the expanded metal 2 is thus designed to have the board thickness X of 0.8 mm or less, the processing of the expanded metal 2 and the attachment thereof to the core section 1a are facilitated and, further, the cost of the expanded metal 2 can be reduced.

Notwithstanding the above, the board thickness X of the expanded metal 2 is not limited to the range of 0.5 mm and more and 0.8 mm and less, and can be changed appropriately in accordance with usages, conditions in use, or so forth of the porous body 10. For instance, the board thickness X can be in the range of 0.3 mm or more and 1.0 mm or less. When the porous body 10 is used not as the part of the inflator for the airbag, but under environment not exposed to high temperature and high pressure, the expanded metal 2 having the board thickness X below 0.5 mm can be employed. Further, when the porous body 10 is used under more harsh environment in terms of pressure and temperature, the expanded metal 2 having the board thickness X over 0.8 mm can be employed.

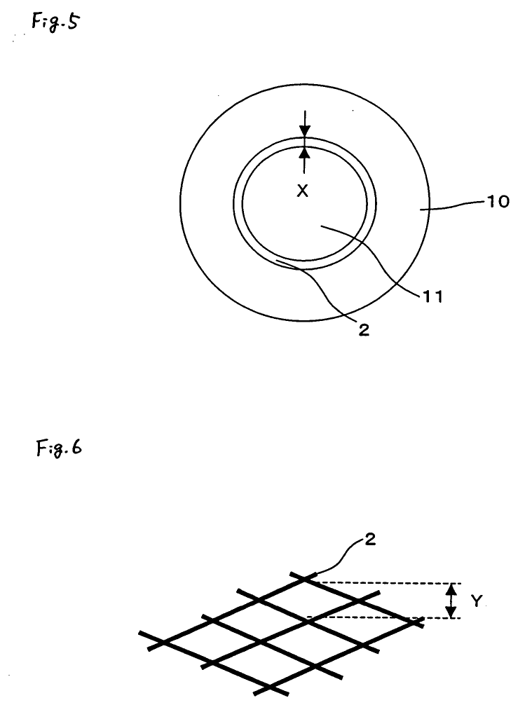

FIG. 6 is a view showing a part of the expanded metal 2.

Generally, expanded metals are classified into a standard type that has rhombic holes (meshes) and a grating type that has hexagonal meshes. In this embodiment, the expanded metal 2 of the former type is employed. A center distance Y corresponding to the shorter diagonal of the rhombic mesh of the expanded metal 2 is approximately 0.5 mm. If the shorter center distance Y is designed to be 0.5 mm, the iron particle 3 having 1.0 mm in diameter and used in this embodiment cannot pass therethrough. Besides, when the porous body 10 is used for example as the part of the inflator for the airbag, the porous body 10 does not hinder air permeability when the blast passes through from the inside toward the outward direction of the porous body 10.

It should be noted that the center distance Y is not limited to 0.5 mm. The center distance Y of the expanded metal 2 can be changed appropriately in accordance with the diameter of the iron particles 3 composing the porous body 10 and use conditions of the porous body 10. If the center distance Y is below 0.3 mm, when the porous body 10 is used for example as the part of the inflator for the airbag, the air permeability in the case where the blast passes therethrough deteriorates. Meanwhile, if the center distance Y is over 2.0 mm, the expanded metal 2 requires customization. In addition, the iron particles 3 for use when the porous body 10 is used as the part of the inflator for the airbag rarely over 2.0 mm in diameter, so that a problem of the iron particles 3 passing through the porous body 10 tends to arise. In consideration of air permeability, cost, and leakage of the iron particles 3, it is therefore preferable to adopt the expanded metal 2 with meshes each having the center distance Y of the shorter diagonal within the range of 0.3 mm or more and 2.0 mm or less.

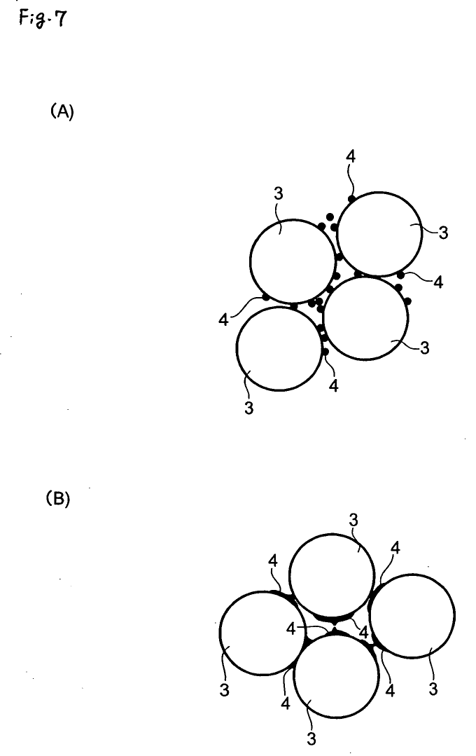

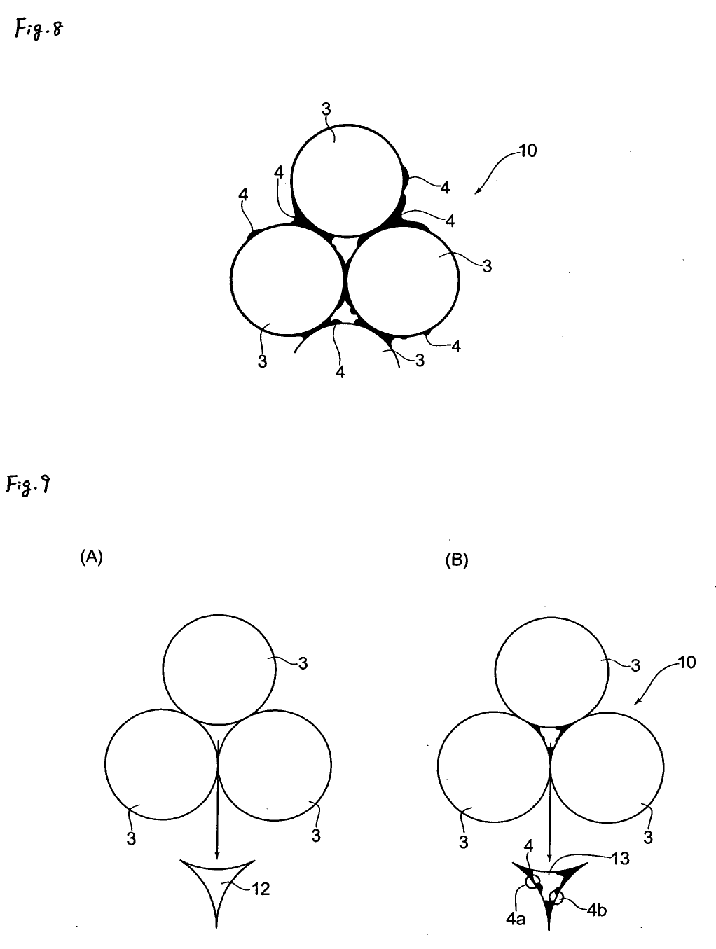

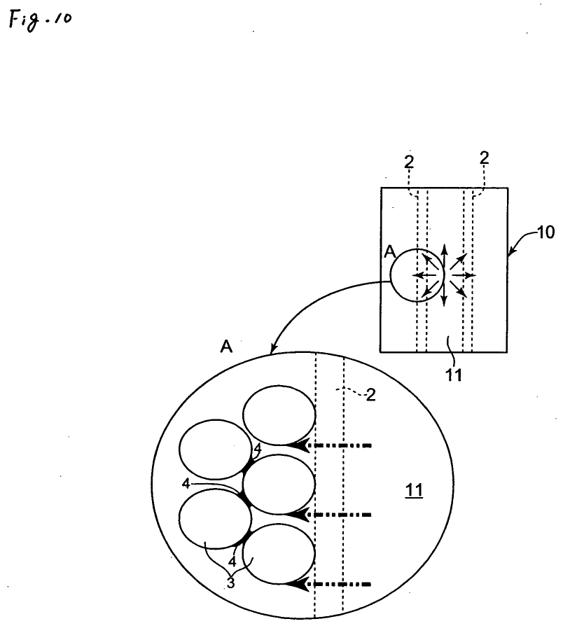

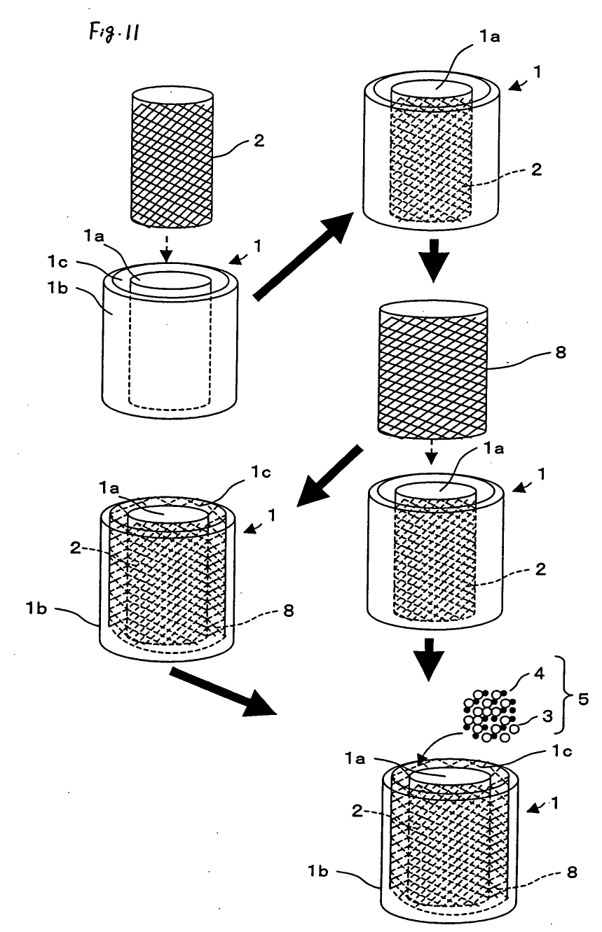

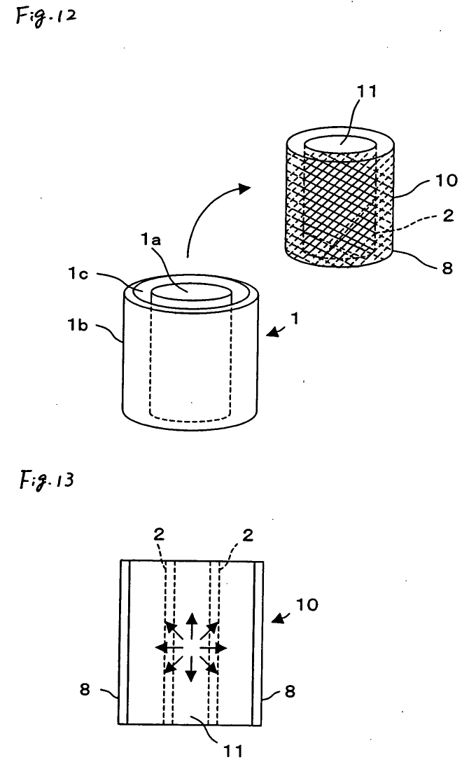

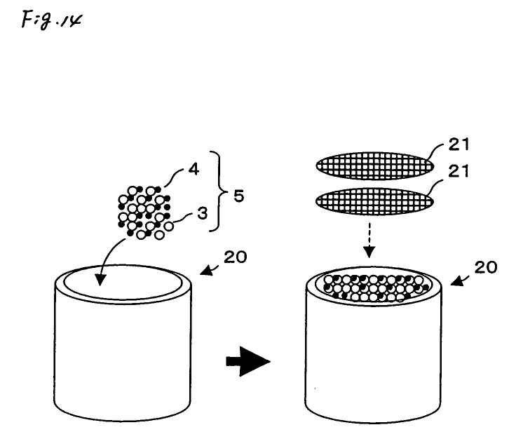

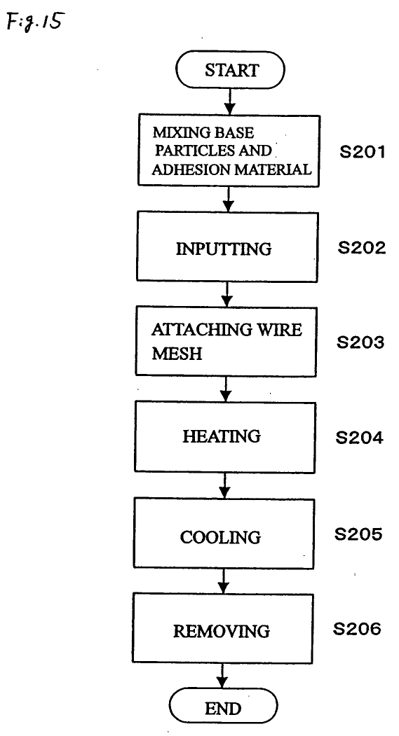

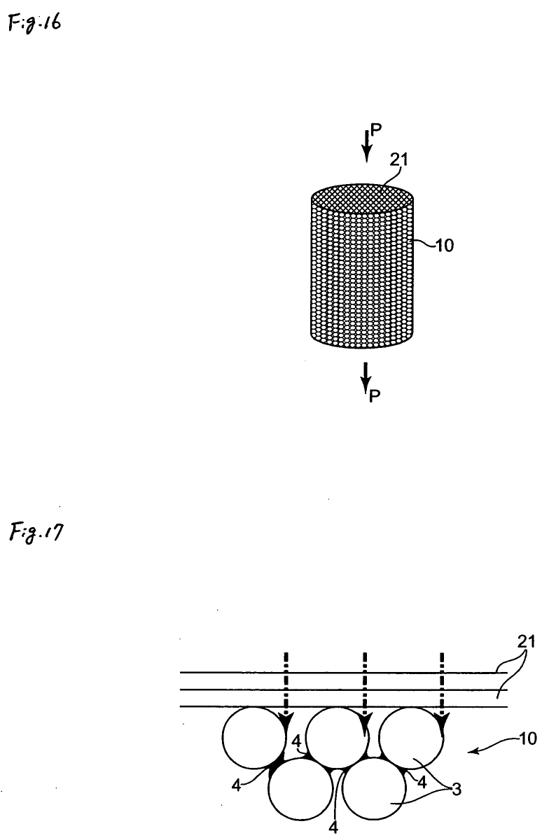

FIG. 7A and FIG. 7B are views schematically showing a change in the copper brazing material 4 that exists between the iron particles 3 in the heating step (step S104) shown in FIG. 3.

As shown in FIG. 7A, before the heating, the copper powders mixed in a liquid flux exist in spaces between the iron particles 3 still in their powdery state. Note that FIG. 7A restrains to illustrate the flux. By mixing the iron particles 3 and the copper brazing material 4 containing the copper powders sufficiently, the copper powders in the copper brazing material 4 come to exist around the iron particles 3 substantially equally. When heating up the mixture 5 of this state, the copper powders transform from a solid state into a molten state and finally into a liquid state to move into the contact boundary of the iron particles 3, as shown in FIG. 7B. This is caused by a capillary action which is a well-known action frequently arises in brazed portions. Meanwhile, the flux evaporates when heated to be a state not to attach to the surfaces of the iron particles 3.

Incidentally, the same reference numeral “4” will be used for denoting copper materials hereinbelow without regard to the form. As previously mentioned, the liquidized copper 4 causes a kind of the capillary action to thereby willingly enter into contact portions (having the smallest spaces) of the iron particles 3. FIG. 7B illustrates a scene where, in almost center of the space formed by four iron particles 3, the coppers 4 exist on the surfaces of the upper and lower iron particles 3 attempt to enter into the contact portions (or adjacent portions) of the iron particles 3.

FIG. 8 is a view schematically showing, in an enlarged illustration, an existing state of the iron particles 3 and the copper 4 of the porous body 10 obtained after the cooling step (step S105) shown in FIG. 4. FIG. 9A and FIG. 9B are views comparatively showing the microstructure (FIG. 9A) of a sintered compact formed only by the iron particles 3 and the microstructure (FIG. 9B) of the porous body 10 of the first embodiment formed by welding the iron particles 3 with the copper 4.

As shown in FIG. 8, the melted copper 4 firmly welds the iron particles 3 to one another by intervening into between the boundary faces of the iron particles 3 while partially covering the surfaces of the iron particles 3. The copper 4 gathers most in the vicinity of the contact portions (or the most adjacent portions) of the iron particles 3. Such a welding gives the porous body 10a tensile strength approximately not of the copper but of the iron.

As shown in FIG. 9B, the porous body 10 according to the first embodiment is structured to have the copper 4 intervening into a space 13 between the iron particles 3. The comparison of the space 13 shown in FIG. 9B with a space 12 shown in FIG. 9A indicates that the space 13 has a protrusion and recession 4a formed by the copper 4, and a protrusion and recession 4b formed by the copper 4 and the surfaces of the iron particles 3. Hence, it is easily found that the surface area of the space 13 is larger than that of the space 12 having no protrusion and recessions 4a, 4b.

In addition, in such a porous body 10 that makes use of the space between the iron particles 3 as an air hole, by increasing a surface area to volume ratio of the space, it is possible to improve cooling capability of a gas passing through the porous body 10. Further, with the increase in the surface area of the space, it is also possible to improve trapping capability of coarse particles and the like included in the porous body 10 (dust collection capability). Similarly, sound-deadening capability is improved. Furthermore, the porous body 10 is produced by firmly welding the iron particles 3 to one another with the copper 4, so that the porous body 10 with higher pressure resistance can be obtained at lower cost, as compared to the porous body formed by sintering merely the iron particles 3 by a solid-phase diffusion.

FIG. 10 is a view showing how a blast arising from an explosion in the hole 11 of the porous body 10 passes through the space inside the porous body 10 together with a partial enlarged illustration.

The blast arising from the explosion in the hole 11 of the porous body 10 passes through the mesh of the expanded metal 2 first. When the porous body 10 is used for example as the part of the inflator for the airbag, the internal temperature at the time of the explosion reaches approximately some 2000° C. Yet, the exposure of the expanded metal 2 to such a high temperature is for an extremely short time, only for approximately 0.004 second, eliminating the problem that the expanded metal 2 itself melts down.

In the meantime, the blast passes through the mesh of the expanded metal 2 and also through the spaces between the iron particles 3 composing the porous body 10 to go away from the porous body 10. After passing through the mesh of the expanded metal 2, the blast contacts the iron particles 3 and the copper 4. Therefore, the temperature of the blast at the time when the blast contacts the iron particles 3 and the copper 4 lowers everytime it passes through the mesh of the expanded metal 2. Accordingly, the cases where the copper 4 is exposed directly to the high temperature reduce, so that a clogging in the vicinity of the internal surface of the hole 11 of the porous body 10 due to the melted copper 4 can be reduced.

### Second Embodiment

Secondly, a second embodiment of the porous body and the method for producing the same according to the present invention will be described. As with the first embodiment, base particles composing the porous body and an adhesion material for causing the base particles to adhere to one another are iron particles 3 and a copper brazing material 4, respectively.

FIG. 11 is a view showing those steps of the production process of the porous body before a heating step. FIG. 12 is a view showing a removing step to take out a porous body being a molded product from a container after the heating step.

The production process of the porous body according to the second embodiment is the same as described in the first embodiment based on FIG. 4. The second embodiment differs from the first embodiment in that, in the attachment step (step S101), not only an expanded metal 2 is disposed to surround a core section 1a, but also an expanded metal 8 is disposed to be in contact with the internal surface of an outer side section 1b of a bottomed cylindrical sleeve container 1.

Hereinbelow, the steps from the attachment step S101 through an inputting step S103 will be described briefly. As shown in FIG. 11, the expanded metal 2 is put to cover the core section 1a of the bottomed cylindrical sleeve container 1. Then, the expanded metal 8 is disposed to contact the internal surface of the outer side section 1b of the bottomed cylindrical sleeve container 1. The expanded metal 8 has a larger diameter than that of the expanded metal 2. In a mixing step S102, the iron particles 3 and the copper brazing material 4 are mixed by an unshown mixing machine. In the inputting step S103, a mixture 5 of the iron particles 3 and the copper brazing material 4 is inputted into the bottomed cylindrical sleeve container 1 having two expanded metals 2, 8 arranged therein.

It should be noted that it is alternatively possible to mix the iron particles 3 and the copper brazing material 4 in the bottomed cylindrical sleeve container 1 after the inputting of the iron particles 3 and the copper brazing material 4 into the bottomed cylindrical sleeve container 1 having two expanded metals 2, 8 arranged therein. The production process of the porous body in that case is shown in FIG. 4, yet, in which the step S102 and the step S103 are replaced with each other.

The heating step S104 and the cooling step S105 are performed using a heating furnace 6 shown in FIG. 2, as in the case of the first embodiment, therefore overlapped description will be restrained.

FIG. 12 is a view showing the removing step for taking out the porous body being the molded product from the container after the heating step.

As shown in FIG. 12, on the inside wall of a hole 11 of the removed porous body 10, the expanded metal 2, which is previously set so as to surround the core section 1a of the bottomed cylindrical sleeve container 1, is fixed. Further, on the lateral surface of the porous body 10, the expanded metal 8, which is previously disposed on the internal surface of the outer side section 1b of the bottomed cylindrical sleeve container 1, is fixed. The two expanded metals 2, 8 are disposed to lightly contact the core section 1a and the internal surface of the outer side section 1b of the container, respectively, so that the porous body 10 can be removed from the bottomed cylindrical sleeve container 1 together with the expanded metals 2, 8 with ease. Accordingly, removing work of the porous body 10 is facilitated, and further, a possible damage that the porous body 10 is to suffer in the production process can be reduced since no unnecessary force is required when removing the porous body 10.

FIG. 13 is a view showing how a blast arising from an explosion in the hole 11 of the porous body 10 passes through inside spaces of the porous body 10.

The operation and effect brought about by the presence of the expanded metal 2 is the same as described in the first embodiment, therefore, such description will be restrained here, and only the operation and effect brought about by the presence of the expanded metal 8 will be described. The expanded metal 8 surrounds the lateral surface of the porous body 10. Accordingly, when the porous body 10 is used as a part of an inflator for an airbag, the porous body 10 is reinforced against the explosion inside the hole 11 to be readily unbreakable.

### Third Embodiment

A sleeve type porous body 10 and the method for producing the same have been described in the above, however, in addition to such a porous body, a solid columnar porous body can also be produced as will be described below.

FIG. 14 is a view for explaining a part of the production process of the solid columnar porous body. FIG. 15 is a flowchart showing the production process of the solid columnar porous body.

The production process of the solid columnar porous body includes: a mixing step (step S201) for mixing an iron particles as base particles and a copper brazing material as an adhesion material, an inputting step (step S202) for inputting the mixed materials into a cylindrical container 20, an attachment step (step S203) for attaching an expanded metal as an aspect of a wire mesh, a heating step (step S204) for inputting the cylindrical container 20 having the mixed materials therein into a heating furnace and heating it, a cooling step (step S205) for cooling a molded product obtained from the heating step, and a removing step (step S206) for taking out the molded product from the cylindrical container 20.

In the mixing step S201, iron particles 3 and a copper brazing material 4 are mixed by an unshown mixing machine. In the inputting step S202, a mixture 5 of the iron particles 3 and the copper brazing material 4 is inputted into the cylindrical container 20. In the attachment step S203, two discoid expanded metals 21, 21 are placed on the mixture 5 in the cylindrical container 20. At the time, a weight may be placed on the expanded metal 21. Further, one or three or more expanded metal(s) 21 may also be acceptable.

It should be noted that the iron particles 3 and the copper brazing material 4 can be alternatively inputted into the cylindrical container 20 first, and mixed thereafter still in the cylindrical container 20. The production process in that case comes to a flow that replaces step S201 and step S202 in FIG. 15 with each other.

The heating step S204 and the cooling step S205 are performed using a heating furnace 6 shown in FIG. 2, as in the case of the first embodiment, therefore overlapped description will be restrained.

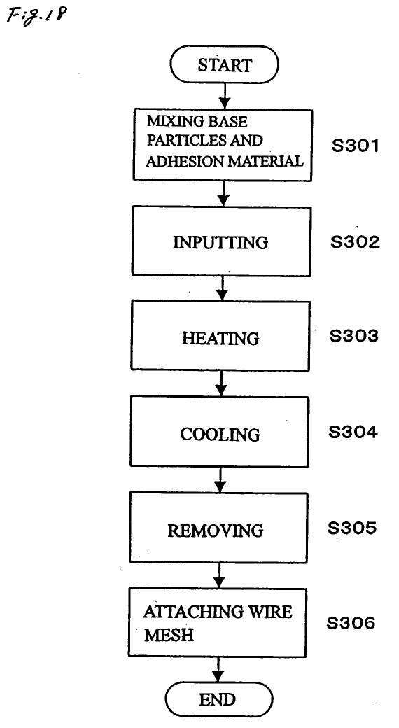

FIG. 16 is a view showing the solid columnar porous body 10 obtained through the production process shown in FIG. 15. FIG. 17 is a view showing, in a partially enlarged illustration, how a blast passes through from one planar surface of the solid columnar porous body 10.

This porous body 10 is formed to have the expanded metals 21, which were placed on the mixture 5, fixedly attached on the upper surface thereof. When this porous body 10 is used as a part of an inflator for an airbag, as shown by arrows P in FIG. 16, the blast passes through the porous body 10 downward from the top. Since there exist the expanded metals 21 on the surface that the blast contact the porous body 10 as shown in FIG. 17, the heat of the blast is cooled down when passing through the expanded metals 21. Therefore, a risk that the copper 4 welding the iron particles 3 composing the porous body 10 might be melted down can be reduced.

It should be noted that the expanded metal 21 may be attached to the porous body 10 alternatively after the cooling step instead of being set before the heating step.

FIG. 18 is a flowchart showing the production process when the expanded metals 21 are attached to the porous body 10 after the cooling step.

As shown in the flowchart in FIG. 18, the production process when the expanded metals 21 are attached to the porous body 10 after the cooling step includes: a mixing step (step S301) for mixing the iron particles as the base particles and the copper brazing material as the adhesion material, an inputting step (step S302) for inputting the mixed materials into the cylindrical container 20, a heating step (step S303) for inputting the cylindrical container 20 having the mixed materials therein into the heating furnace and heating it, a cooling step (step S304) for cooling the molded product obtained from the heating step, and a removing step (step S305) for taking out the molded product from the cylindrical container 20, and an attachment step (step S306) for attaching the expanded metal 21 to one planar surface of the porous body 10. Note that the attachment of the expanded metal 21 in the attachment step may be realized in any manner such as the welding and plating.

In the above, the respective embodiments of the porous body and the method for producing the same according to the present invention have been described. However, the present invention is not limited to the above embodiments and may be embodied in various modified embodiments.

For instance, the combination of the base particle and the adhesion material is not limited to iron and copper. Any combination such as of iron and nickel base alloy (for example, nickel brazing material), stainless steel and copper, and so forth are applicable at will. Specifically, as a base particle, other than iron, carbon steel, alloy steel, stainless steel, copper, aluminum, cemented carbide, and so forth are applicable. Meanwhile, as an adhesion material, various materials such as copper, nickel, silver, phosphor copper are selectable, provided that, as a requirement, the adhesion material melts better than the base particle under a high temperature, as previously stated.

Further, in the above embodiments, for the base particle and the adhesion material, metals are used, and in which the base particles are welded by the adhesion material. Notwithstanding the above, when performing an adhesion, as a superior concept, instead of the welding, the base particle may be a ceramic and the adhesion material may be a metal. Further, the base particle may be a metal or a ceramic and the adhesion material may be a resin. Furthermore, both the base particle and adhesion material may be ceramics.

In the above respective embodiments, the porous body 10 has a cylindrical shape or a solid columnar shape, however, the porous body may have a cylindrical shape or solid columnar polygonal shape with its both ends being polygonal. When producing the cylindrical porous body having polygonal both ends, a core section of the porous body may have either a columnar shape or a polygonal columnar shape. An expanded metal to be disposed around the core section may have any shape in accordance with the shape of the core section. Further, the expanded metal to be disposed on the inside wall of the outer side of a cylindrical molding container may have any shape in accordance with the shape of the inside wall of the outer side thereof. When producing the solid columnar polygonal porous body, the container may have a polygonal cylindrical shape, and the expanded metal to be placed on the mixture of the base particles and the adhesion material may have any shape in accordance with a polygonal opening of the cylindrical container.

Furthermore, a wire mesh other than the expanded metal, for example, a balled up thin metal wire, a knitted lattice metal wire, or so forth can be employed.

The porous body according to the present invention is suitable for a part of an inflator for a vehicle airbag. Also, the porous body according to the present invention is applicable to a filter making use of spaces between base particles as an air hole or a water permeable hole, or an absorbent material or a sound-deadening material making use of larger surface areas of the spaces between the base particles.

1. A cylindrical porous body comprising a number of base particles caused to adhere to one another by an adhesion material having a lower melting point than the melting point of said base particles,
wherein a wire mesh is attached to an internal surface of said porous body.

2. The porous body according to claim 1,
wherein said wire mesh is attached to a lateral surface of said porous body.

3. The porous body according to claim 1 or claim 2,
wherein the diameter of a wire of said wire mesh is within the range of 0.3 mm or more and 1.0 mm or less.

4. The porous body according to claim 1 or claim 2,
wherein the diameter of a wire of said wire mesh is within the range of 0.5 mm or more and 0.8 mm or less.

5. The porous body according to claim 1 or claim 2,
wherein said wire mesh is an expanded metal.

6. The porous body according to claim 5,
wherein the expanded metal has a number of rhombic holes and a shorter diagonal of the diagonals of the hole has a length passing none of said base particle therethrough and within the range of 0.3 mm or more and 2.0 mm or less.

7. The porous body according to claim 1 or claim 2,
wherein said adhesion material is metal.

8. The porous body according to claim 1 or claim 2,
wherein said base particle is iron and said adhesion material is copper.

9. A method for producing a porous body, comprising the steps of:
mixing a number of base particles composing the porous body, and an adhesion material causing the base particles to adhere to one another, the adhesion material having a lower melting point than the melting point of the base particle;
attaching a wire mesh to a lateral surface of a central axis of a cylindrical molding container for molding to form a cylindrical shape.
heating a mixture, which is obtained by said mixing step, in a state being in the cylindrical molding container after said attachment step so that the base particles adhere to one another with the adhesion material, and
removing a molded product from the cylindrical molding container.

10. The method for producing the porous body according to claim 9,
wherein said attachment step of the wire mesh is the step of attaching the wire mesh also to an internal surface of the cylindrical molding container.

11. The method for producing the porous body according to claim 9,
wherein the diameter of a wire of the wire mesh is within the range of 0.3 mm or more and 1.0 mm or less.

12. The method for producing the porous body according to claim 9,
wherein the diameter of a wire of the wire mesh is within the range of 0.5 mm or more and 0.8 mm or less.

13. The method for producing the porous body according to claim 9,
wherein the wire mesh is an expanded metal.

14. The method for producing the porous body according to claim 13,
wherein the expanded metal has a number of rhombic holes and a shorter diagonal of the diagonals of the hole has a length passing none of the base particle therethrough and within the range of 0.3 mm or more and 2.0 mm or less.

15. The method for producing the porous body according to claim 9,
wherein the adhesion material is metal.

16. The method for producing the porous body according to claim 9,
wherein the base particle is iron and the adhesion material is copper.
he method for producing the porous body according to claim 8 or claim 9,
wherein the diameter of a wire of the wire mesh is within the range of 0.3 mm or more and 1.0 mm or less.

17. The method for producing the porous body according to claim 8,
wherein the diameter of a wire of the wire mesh is within the range of 0.5 mm or more and 0.8 mm or less.

18. The method for producing the porous body according to claim 8,
wherein the wire mesh is an expanded metal.

19. The method for producing the porous body according to claim 18,
wherein the expanded metal has a number of rhombic holes and a shorter diagonal of the diagonals of the hole has a length passing none of the base particle therethrough and within the range of 0.3 mm or more and 2.0 mm or less.

20. The method for producing the porous body according to claim 8,
wherein the adhesion material is metal.

21. The method for producing the porous body according to claim 8,
wherein the base particle is iron and the adhesion material is copper.
